(19) United States
(12) Patent Application Publication
(10) Pub. No.: US 2005/0124658 A1
(43) Pub. Date: Jun. 9, 2005

(54) Decahydronaphtho[2,3-c]furan derivatives

(75) Inventors: Masanori Takadoi, Kuki-shi (JP); Shiro Terashima, Tokyo (JP)

Correspondence Address: OBLON, SPIVAK, MCCLELLAND, MAIER & NEUSTADT, P.C., 1940 DUKE STREET, ALEXANDRIA, VA, 22314, US

(73) Assignees: Segami Chemical Research Center; Kyorin Pharmaceutical Co., Ltd.

(21) Appl. No.: 10/498,814

(22) PCT Filed: Dec. 20, 2002
(86) PCT No.: PCT/JP02/13344

(30) Foreign Application Priority Data
Dec. 25, 2001 (JP) 2001-390999

(51) Int. Cl. C07D 493/04; A61K 31/452; C07D 45/02

(52) U.S. Cl. 514/320; 546/196; 549/456; 549/458

(57) ABSTRACT

The invention provides useful novel analogues of himbacine, being an alkaloid with potent and selective antagonism against muscarine M₂ receptor.
Hydronaphtho [2,3-c] furan derivatives represented by a following general formula (1) 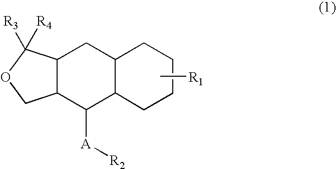 and their acid addition salts.

### TECHNICAL FIELD

The present invention relates to decahydronaphtho[2,3-c]furan derivatives being novel analogues of himbacine that exhibits potent and selective antagonism against muscarine M₂ receptor and is expected to be used as a therapeutic drug for Alzheimer's disease, and preparative processes thereof.

### BACKGROUND TECHNOLOGIES

Himbacine ((+)-himbacine) is a piperidine alkaloid which was isolated from Galbulimima baccata being a kind of pinaceous plants and the structure of which was determined in 1956. As the structural features thereof, three points can be mentioned; a point that 5-membered ring lactone is condensed to thermodynamically stable trans-decalin ring by cis configuration, a point that it has 8 asymmetric centers including 4 internuclear hydrogens, and further a point that 3-ring portion and piperidine ring are bound via trans double bond.

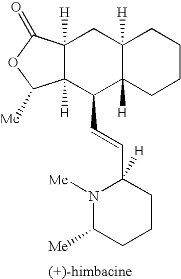

In recent years, senile dementia represented by Alzheimer's type dementia has posed a significant problem socially and an essential therapeutic drug therefor is desired earnestly. As one of approaches therefor, from a phenomenon of hypergasia of central cholinergic nerve in dementia patient, the development of therapeutic drugs based on so-called “choline hypothesis” is being made actively. Roughly classifying them, they can be divided into following four; (1) inhibitor for taking-in of choline, (2) inhibitor for acetylcholinesterase, (3) activator for synthesizing choline acetyltransferase, and (4) acting drug on muscarine receptor (muscarine M₁ agonist or antagonist of M₂ receptor). It has become clear lately that himbacine exhibits an inhibitory action potently and selectively against M₂ receptor that is considered to have suppressive work for the exudation of acetylcholine in nerve terminal, leading to the finding of possibility as an antidemential drug. As described, himbacine is a compound attracting an attention worldwide from two points of potent activity and interesting chemical structure, and, in recent years, total synthesis that uses intramolecular Diels-Alder reaction as a key reaction and derivative synthesis are reported from some groups (Kozikowski et al, total synthesis: a) J. Am. Chem. Soc. 1995, 117, 9369-9370. b) J. org. Chem. 1997, 62, 5023-5033. derivative synthesis: a) Bioorg. Med. Chem. Lett. 1992, 2, 797-802. b) Bioorg. Med. Chem. Lett. 1993, 3, 1247-1252. c) Bioorg. Med. Chem. Lett. 1995, 5, 61-66. Chackalamannil et al, total synthesis: a) J. Am. Chem. Soc. 1996, 118, 9812-9813. b) J. Org. Chem. 1999, 64, 1932-1940. derivative synthesis: Bioorg. Med. Chem. Lett. 1999, 9, 901-906, etc.). Moreover, with respect to the analogues of himbacine, a following patent by Schering group that uses intramolecular Diels-Alder reaction as a key reaction is disclosed (Chackalamannil et al, WO9926943). Therein, compounds wherein R₁₁ and R₁₂ being 3-position substituents of himbacine in following general formula are hydrogen atoms together are claimed, but only heteroaromatic ring wherein “Het” in the figure is monocyclic, bicyclic or tricyclic is claimed. Furthermore, in said patent, no concrete examples for preparing compounds wherein R₁₁ and R₁₂ are hydrogen atoms together are disclosed. Moreover, said patent is disclosed as compounds with antagonism against thrombin receptor, and the antagonism against muscarine M₂ subtype receptor as shown in the present invention is not touched at all.

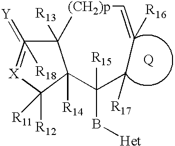

As resemblant compounds to hydronaphtho [2,3-c]furan derivatives shown in claim 4, derivatives shown below are publicly known (Tochtermann et al, Tetrahedron Lett. 1994, 35, 1165-1168). In said reference, only following hydronaphtho[2,3-c]furan derivatives are described and conversion to hydroazulene lactone derivatives is only attempted.

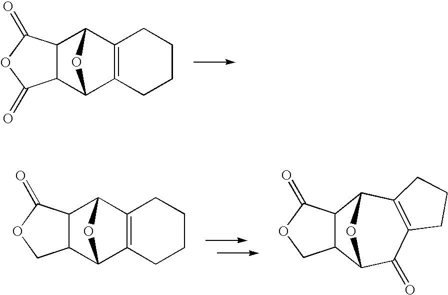

Moreover, by the inventors, total synthesis of himbacine that uses intermolecular Diels-Alder reaction as a key reaction is disclosed (Terashima et al, Jpn. Kokai Tokkyo Koho JP 2000-229961, Takadoi et al, Tetrahedron Lett. 1999, 40, 3399-3402). 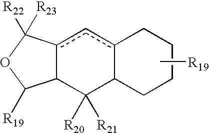
Said patent claims preparative intermediates needed for the synthesis of himbacine, and R₁₉ corresponding to 3-position substituent of himbacine is only claimed to be lower alkyl group or substituted or unsubstituted aralkyl group. Hence, synthesis of the analogues of himbacine with structural characteristics such that 3-position substituent of himbacine is hydrogen atom as shown in the present invention is not yet reported and the antagonism thereof against muscarine M₂ subtype receptor is also not made clear.

The purpose of the present invention is to provide novel analogues of himbacine that is expected to be used as a therapeutic drug for Alzheimer's disease, and preparative intermediates thereof.

### DISCLOSURE OF THE INVENTION

As a result of diligent studies in view of said subjects, the inventors have found that the inventive following compounds have potent and very subtype-selective antagonism against muscarine M₂ receptor, and also have found that they are very useful as preparative intermediates in their preparation, leading to the completion of the invention.

Namely, the invention provides decahydronaphtho[2,3-c]furan derivatives represented by a following general formula (1) 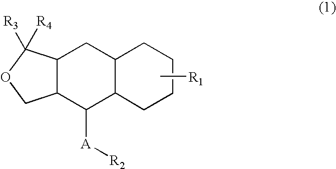
(wherein R₁ denotes a hydrogen atom, lower alkyl group or substituted or unsubstituted aralkyl group, R₂ denotes an aliphatic heterocycle of 5 to 14-membered ring which may contain one or more hetero-atoms such as O, N and S and which may have one or more substituents, or aromatic heterocycle, R₃ and R₄ unitedly denote an oxygen atom, or R₃ denotes a hydrogen atom and R₄ denotes a hydroxyl group, lower alkoxy group, substituted or unsubstituted aralkyloxy group or lower acyloxy group, and A and R₂ unitedly denote following general formula (2) 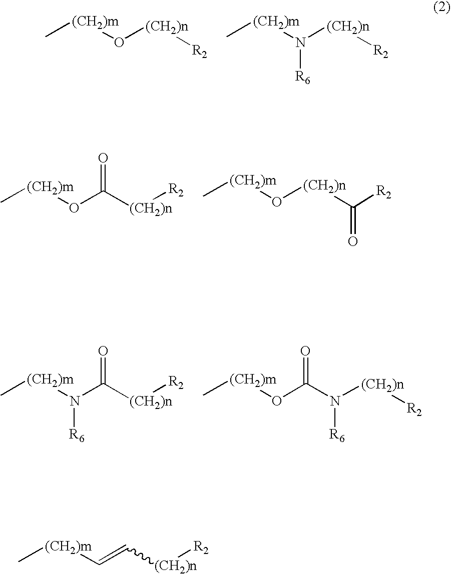
(m and n denote 0, 1 or 2, and R₅ denotes a hydrogen atom, lower alkyl group or substituted or unsubstituted aralkyl group)), and their acid addition salts, decahydronaphtho[2,3-c]furan derivatives represented by a following general formula (1-1) 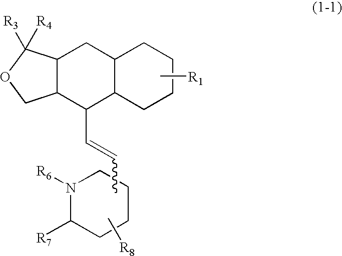
(wherein R₁ denotes a hydrogen atom, lower alkyl group or substituted or unsubstituted aralkyl group, R₃ and R₄ unitedly denote an oxygen atom, or R₃ denotes a hydrogen atom and R₄ denotes a hydroxyl group, lower alkoxy group, substituted or unsubstituted aralkyloxy group or lower acyloxy group, R₆ denotes a hydrogen atom, lower alkyl group, substituted or unsubstituted aralkyl group or protective group of amino group, R₇ denotes a hydrogen atom, lower alkyl group or substituted or unsubstituted aralkyl group, and R₈ denotes a hydrogen atom, lower alkyl group or substituted or unsubstituted aralkyl group), and their acid addition salts, decahydronaphtho[2,3-c]-furan derivatives represented by a following general formula (1-2) 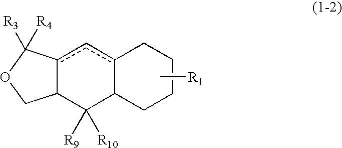
(wherein R₁ denotes a hydrogen atom, lower alkyl group or substituted or unsubstituted aralkyl group, R₃ and R₄ unitedly denote an oxygen atom, or R₃ denotes a hydrogen atom and R₄ denotes a hydroxyl group, lower alkoxy group, substituted or unsubstituted aralkyloxy group or lower acyloxy group, R₉ and R₁₀ unitedly denote an oxygen atom or methylene group, or R₉ denotes a hydrogen atom and R₁₀ denotes a hydroxyl group, lower alkoxy group, substituted or unsubstituted aralkyloxy group or lower acyloxy group, hydroxymethyl group which may be substituted with oxygen atom, substituted or unsubstituted arylsulfenylmethyl group or substituted or unsubstituted arylsulfonylmethyl group, and, in the case of broken lines accompanied, one denotes single bond and the other denotes double bond, or both denote single bonds), and hydronaphtho[2,3-c]furan derivatives represented by a following general formula (3) 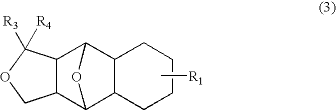
(wherein R₁ denotes a hydrogen atom, lower alkyl group or substituted or unsubstituted aralkyl group, and R₃ and R₄ unitedly denote an oxygen atom, or R₃ denotes a hydrogen atom and R₄ denotes a hydroxyl group, lower alkoxy group, substituted or unsubstituted aralkyloxy group or lower acyloxy group), and their preparative processes.

In the invention, for “lower alkyl groups”, straight chain or branched ones with carbon atoms of 1 to 6 such as methyl, ethyl, 1-methylethyl, 1,1-dimethylethyl, propyl and 2-methylpropyl are mentioned and it doesn't matter whether they are saturated or unsaturated. For “aralkyl group”, benzyl group, 1-phenylethyl group, etc. are mentioned and, as substituents, lower alkyl group, lower alkoxy group, halogen atom, cyano group, nitro group, etc. are mentioned. For “lower alkoxy groups”, straight chain or branched ones with carbon atoms of 1 to 6 such as methoxy, ethoxy, 1-methylethoxy, 1,1-dimethylethoxy, propoxy and 2-methylpropoxy are mentioned and it doesn't matter whether they are saturated or unsaturated. For “aralkyloxy group”, benzyloxy group, 1-phenylethoxy group, etc. are mentioned and, as substituents, lower alkyl group, lower alkoxy group, halogen atom, cyano group, nitro group, etc. are mentioned. For “lower acyloxy groups”, ones with carbon atoms of 1 to 5 such as formyl group, acetoxy group, propionyloxy group and 2,2-dimethylpropionyloxy group are mentioned. For “hydroxymethyl groups which may be substituted with oxygen atom”, hydroxymethyl group which may be substituted with methylene chain, arylacyloxymethyl group, hydroxymethyl group which formed elimination group unitedly with oxygen atom, etc. are mentioned. For “arylacyl groups” referred to so here, benzoyl group etc. are mentioned and, as substituents lower alkyl group, lower alkoxy group, halogen atom, cyano group, nitro group, etc. arementioned. Moreover, as protective groups of hydroxyl group, trialkylsilyl groups such as trimethylsilyl group and t-butyldimethylsilyl group, arylmethyl groups such as benzyl group and diphenylmethyl group, acyl groups such as acetyl group and propionyl group, lower alkoxymethyl groups such as methoxymethyl group and ethoxymethyl group, aralkyloxymethyl groups such as benzyloxymethyl group, tetrahydropyranyl group, etc. are mentioned, and the introduction and elimination thereof can be performed by appropriately adopting the methods described in the reference (Green, T. W.; Wuts, P. G. M. “Protective Groups in organic Synthesis”, 2^{nd} Ed., Wiley Interscience Publication, John-Wiley & Sons, New York, 1991, pp 14-118). Moreover, for “elimination groups united with oxygen atom”, for example, lower alkylsulfonyloxy group, arylsulfonyloxy group, etc. are mentioned. For “protective groups of amino group”, for example, lower acyl groups such as acetyl and propionyl, lower alkoxycarbonyl groups such as ethoxycarbonyl and t-butoxycarbonyl, benzyl group, etc. are mentioned, and the introduction and elimination thereof can be performed by appropriately adopting the methods described in the reference (Green, T. W.; Wuts, P. G. M. “Protective Groups in Organic Synthesis”, 2^{nd} Ed., Wiley Interscience Publication, John-Wiley & Sons, New York, 1991, pp 14-118).

For “one or more substituents” in “aliphatic heterocycle of 5 to 14-membered ring which may contain one or more hetero-atoms such as O, N and S and which may have one or more substituents”, for example, halogen atom, lower alkyl group, lower alkoxy group, lower alkylthio group, lower alkoxycarbonyl group, nitro group, amino group, cyano group, etc. are mentioned, and, for “aliphatic heterocycles”, for example, pyrrolidyl, piperidyl, piperazyl, morpholyl, etc. are mentioned. The “amino group” in this case may be substituted with acyl, for example, acetyl etc. or may be substituted with one to two lower alkyl groups. For “aromatic heterocycles”, for example, furanyl, thienyl, pyrazolyl, imidazolyl, oxazolyl, thiazolyl, pyridyl, pyrimidyl, pyridazyl, pyrazyl, etc. are mentioned, and they may also be condensed with benzene ring at arbitrary position. The “amino group” in this case may be substituted with acyl, for example, acetyl etc. or may be substituted with one to two lower alkyl groups. “Acid addition salts” are pharmacologically acceptable salts with, for example, inorganic acids such as hydrochloric acid, acetic acid and sulfuric acid, and organic acids such as citric acid, succinic acid, fumaric acid, maleic acid and tartaric acid.

As preferable compounds in the invention, (3aR,4S,4aS,8aR,9aS)-decahydro-4-[2-(E)-[(2R,6S)-6-methylpiperidine-2-yl]ethenyl]naphtho[2,3-c]furan-1(3H)-one, (3aR,4S,4aS,8aR,9aS)-decahydro-4-[2-(E)-[(2S,6R)-6-methylpiperidine-2-yl]ethenyl]naphtho[2,3-c]furan-1(3H)-one, (3aS,4R,4aR,8aS,9aR)-decahydro-4-[2-(E)-[(2R,6S)-6-methylpiperidine-2-yl]ethenyl]naphtho[2,3-c]furan-1(3H)-one, (3aS,4R,4aR,8aS,9aR)-decahydro-4-[2-(E)-[(2S,6R)-6-methylpiperidine-2-yl]ethenyl]naphtho[2,3-c]furan-1(3H)-one, and (3aR,4S,4aS,8aR,9aS)-decahydro-4-[2-(E)-[(2R,6S)-1,6-dimethylpiperidine-2-yl]ethenyl]naphtho[2,3-c]furan-1(3H)-one,(3aR,4S,4aS,8aR,9aS)-decahydro-4-[2-(E)-[(2S,6R)-1,6-dimethylpiperidine-2-yl]ethenyl]naphtho[2,3-c]furan-1(3H)-one,(3aS,4R,4aR,8aS,9aR)-decahydro-4-[2-(E)-[(2R,6S)-1,6-dimethylpiperidine-2-yl]ethenyl]naphtho[2,3-c]furan-1(3H)-one,(3aS,4R,4aR,8aS,9aR)-decahydro-4-[2-(E)-[(2S,6R)-1,6-dimethylpiperidine-2-yl]ethenyl]naphtho[2,3-c]furan-1(3H)-one, etc. are mentioned.

Besides, the compounds of the invention have a plurality of asymmetric carbons, hence corresponding optical isomers can exist, but these optical isomers and their mixtures are to be included in the invention.

A group of the compounds represented by the general formula (1) of the invention can be prepared, for example, according to following preparative processes, using the compounds represented by said general formula (3) for key intermediates. 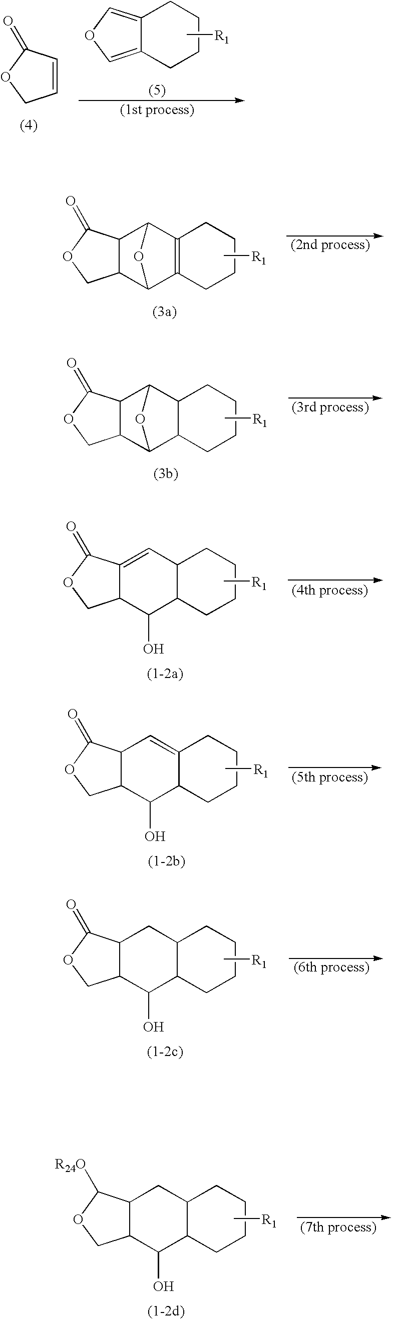 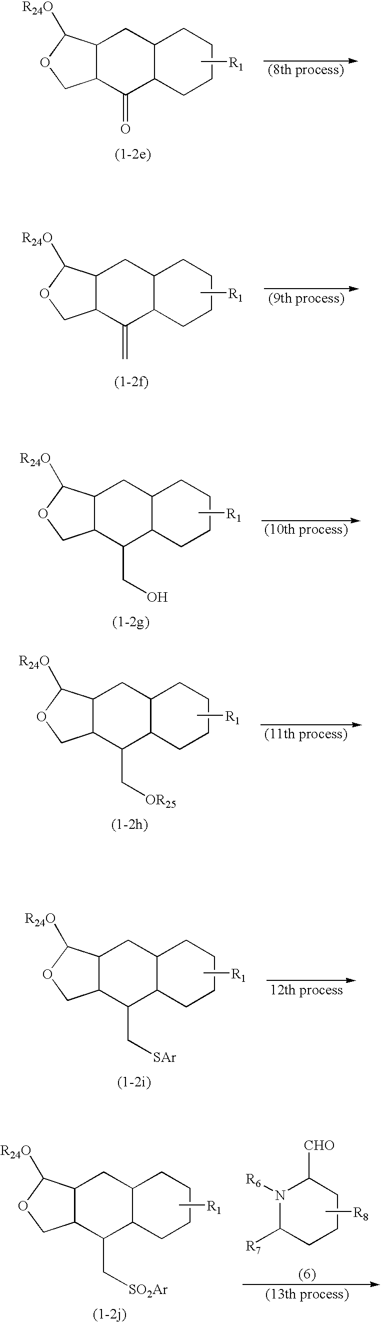 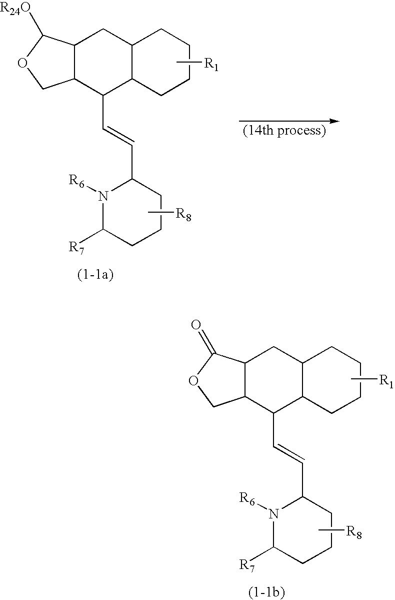
(First Process)

In this process, 4,5,6,7-tetrahydroisobenz ofuran represented by said general formula (5) is added to 2(5H)-furanone represented by said general formula (4) to prepare 4,9-epoxyoctahydronaphtho [2,3-c]furan-1(3H)-one derivatives represented by said general formula (3a).

This reaction can be performed in the presence or absence of Lewis acid such as zinc chloride, zinc bromide, zinc iodide, boron trifluoride, aluminum chloride, tin tetrachloride, boron trifluoride-diethyl ether complex or lithium perchlorate, rhodium complex such as Wilkinson's complex, sodium dodecyl sulfate, or cetyltrimethylammonium bromide. The reaction is conducted in the presence or absence of, for example, hydrocarbonic solvent such as pentane, hexane, cyclohexane, benzene, toluene or xylene, halogenated hydrocarbonic solvent such as dichloromethane, 1,2-dichloroethane, chloroform or carbon tetrachloride, ethereal solvent such as diethyl ether, tetrahydrofuran or 1,4-dioxane, aprotic polar solvent such as acetonitrile, propionitrile, nitromethane, nitroethane, N,N-dimethylformamide or dimethyl sulfoxide, or mixed solvent thereof with water, and proceeds smoothly usually at −20° C. to 200° C. Moreover, as the case may be, a stabilizer like radical scavenger such as 2,6-di-t-butyl-4-methylphenol(BHT), or the like may be added.

(Second Process)

In this process, the double bond in 4,9-epoxyoctahydronaphtho[2,3-c]furan-1(3H)-one derivatives represented by said general formula (3a), obtainable in said first process, is reduced catalytically to prepare 4,9-epoxydecahydronaphtho[2,3-c]furan-1(3H)-one derivatives represented by said general formula (3b).

This reaction is performed usually in a solvent using a catalyst such as palladium-carbon, Raney nickel, palladium hydroxide, rhodium-alumina or platinum oxide. For the solvent, any can be used, if it does not take part in the reaction, but, for example, hydrocarbonic solvent such as pentane, hexane, cyclohexane, benzene, toluene or xylene, halogenated hydrocarbonic solvent such as dichloromethane, 1,2-dichloroethane, chloroform or carbon tetrachloride, ethereal solvent such as diethyl ether, tetrahydrofuran or 1,4-dioxane, alcoholic solvent such as methanol, ethanol, 1-propanol, 2-propanol, 1-butanol, 2-butanol, 2-methyl-1-propanol or 2-methyl-2-propanol, or mixed solvent thereof with water is used suitably. The reaction proceeds smoothly at 0° C. to 100° C. and at 98.1 KPa to several hundreds of KPas.

(Third Process)

In this process, the ether bond of 4,9-epoxydecahydronaphtho [2,3-c]furan-1(3H)-one derivatives represented by said general formula (3b), obtainable in said second process, is cleaved to prepare octahydronaphtho[2,3-c]furan-1(3H)-one derivatives represented by said general formula (1-2a).

This reaction can be performed usually in the presence of suitable reacting agent, for example, alkali metal alkoxide such as sodium methoxide or sodium ethoxide, alkali metal organic base such as n-butyllithium, lithium bis(trimethylsilyl)amide, sodium bis(trimethylsilyl)amide or potassium bis(trimethylsilyl)amide, tertiary organic base such as triethylamine, diisopropylethylamine, pyridine, N-methylmorpholine, imidazole, pyrrolidine, piperidine, 1,5-diazabicyclo[4.3.0]non-5-ene or 1,8-diazabicyclo[5.4.0]undec-7-ene, inorganic base such as potassium carbonate or sodium hydrogencarbonate, or Lewis acid such as zinc chloride, zinc bromide, zinc iodide, boron trifluoride, aluminum chloride, tin tetrachloride, boron trifluoride-diethyl ether complex or lithium perchlorate. For the solvent, any can be used, if it does not take part in the reaction, but, for example, hydrocarbonic solvent such as pentane, hexane, cyclohexane, benzene, toluene or xylene, halogenated hydrocarbonic solvent such as dichloromethane, 1,2-dichloroethane, chloroform or carbon tetrachloride, or ethereal solvent such as diethyl ether, tetrahydrofuran or 1,4-dioxane is used suitably. The reaction proceeds smoothly at −110° C. to 100° C.

(Fourth Process)

In this process, the double bond of octahydronaphtho[2,3-c]furan-1(3H)-one derivatives represented by said general formula (1-2a), obtainable in said third process, is isomerized using suitable base to prepare octahydronaphtho[2,3-c]furan-1(3H)-one derivatives represented by said general formula (1-2b).

This reaction can be performed in the presence of, for example, alkali metal alkoxide such as sodium methoxide or sodium ethoxide, alkali metal organic base such as n-butyllithium, lithium bis(trimethylsilyl)amide, sodium bis(trimethylsilyl)amide or potassium bis(trimethylsilyl)amide, tertiary organic base such as triethylamine, diisopropylethylamine, pyridine, N-methylmorpholine, imidazole, pyrrolidine, piperidine, 1,5-diazabicyclo[4.3.0]non-5-ene or 1,8-diazabicyclo[5.4.0]undec-7-ene, or inorganic base such as potassium carbonate or sodium hydrogencarbonate. For the solvent, any can be used, if it does not take part in the reaction, but, for example, hydrocarbonic solvent such as pentane, hexane, cyclohexane, benzene, toluene or xylene, halogenated hydrocarbonic solvent such as dichloromethane, 1,2-dichloroethane, chloroform or carbon tetrachloride, ethereal solvent such as diethyl ether, tetrahydrofuran or 1,4-dioxane, or alcoholic solvent such as methanol, ethanol, 1-propanol, 2-propanol, 1-butanol, 2-butanol, 2-methyl-1-propanol or 2-methyl-2-propanol is used suitably. The reaction proceeds smoothly at −110° C. to 100° C.

(Fifth Process)

In this process, the double bond in octahydronaphtho[2,3-c]furan-1(3H)-one derivatives represented by said general formula (1-2b), obtainable in said fourth process, is reduced catalytically to prepare decahydronaphtho[2,3-c]furan-1(3H)-one derivatives represented by said general formula (1-2c).

This reaction is performed usually in a solvent using a catalyst such as palladium-carbon, Raney nickel, palladium hydroxide, rhodium-alumina or platinum oxide. For the solvent, any can be used, if it does not take part in the reaction, but, for example, hydrocarbonic solvent such as pentane, hexane, cyclohexane, benzene, toluene or xylene, halogenated hydrocarbonic solvent such as dichloromethane, 1,2-dichloroethane, chloroform or carbon tetrachloride, ethereal solvent such as diethyl ether, tetrahydrofuran or 1,4-dioxane, alcoholic solvent such as methanol, ethanol, 1-propanol, 2-propanol, 1-butanol, 2-butanol, 2-methyl-1-propanol or 2-methyl-2-propanol, or mixed solvent thereof with water is used suitably. The reaction proceeds smoothly at 0° C. to 100° C. and at 98.1 KPa to several hundreds of KPas.

(Sixth Process)

In this process, the lactone carbonyl group in decahydronaphtho [2,3-c]furan-1(3H)-one derivatives represented by said general formula (1-2c), obtainable in said fifth process, is reduced and the 1-hydroxyl group of 1,4-dihydroxy-dodecahydronaphtho[2,3-c]furan-1(3H)-one derivatives produced is protected by alkylating selectively to prepare 1-alkoxy-4-hydroxy-dodecahydronaphtho [2,3-c]furan-1(3H)-one derivatives represented by said general formula (1-2d) (R₂₄ denotes a lower alkyl group, substituted or unsubstituted aralkyl group or lower acyl group).

The reduction of this process is performed using dialkyl aluminum hydride such as diisobutyl aluminum hydride. For the solvent, any can be used, if it does not take part in the reaction, but, for example, hydrocarbonic solvent such as pentane, hexane, cyclohexane, benzene, toluene or xylene, halogenated hydrocarbonic solvent such as dichloromethane, 1,2-dichloroethane, chloroform or carbon tetrachloride, or ethereal solvent such as diethyl ether, tetrahydrofuran or 1,4-dioxane is used. The reaction proceeds smoothly at −100° C. to 100° C. The etherification of 1-position hydroxyl group to be conducted successively is conducted in an alcoholic solvent in the presence of suitable Lewis acid. For the “suitable Lewis acid”, for example, zinc chloride, zinc bromide, zinc iodide, boron trifluoride, aluminum chloride, tin tetrachloride, boron trifluoride-diethyl ether complex, lithium perchlorate or the like is mentioned and, for the alcoholic solvent, for example, methanol, ethanol, 1-propanol, 2-propanol, 1-butanol, 2-butanol, 2-methyl-1-propanol, 2-methyl-2-propanol or the like is used suitably. The reaction proceeds smoothly usually at −100° C. to 100° C. Moreover, the introduction of aralkyl group or acyl group to 1-position hydroxyl group is performed also according to the publicly known method (Green, T. W.; Wuts, P. G. M. “Protective Groups inorganic Synthesis”, 2^{nd} Ed., Wiley Interscience Publication, John-Wiley & Sons, New York, 1991, pp 46-66 and pp 87-118).

(Seventh Process)

In this process, the 4-hydroxyl group in 4-hydroxydodecahydronaphtho[2,3-c]furan-1(3H)-one derivatives represented by said general formula (1-2d), obtainable in said sixth process, is oxidized to prepare 4-oxo-decahydronaphtho[2,3-c]furan-1(3H)-one derivatives represented by said general formula (1-2e).

For the oxidizing agent to be used in this process, chromic acid, chromium trioxide-pyridine mixed system, dimethyl sulfoxide-oxalyl chloride-triethylamine mixed system, ruthenium complex, Dess-Martin reagent or the like can be used. The oxidation is usually desirable to be conducted in a solvent and, for example, halogenated hydrocarbonic solvent such as dichloromethane, 1,2-dichloroethane, chloroform or carbon tetrachloride is used. The reaction proceeds smoothly at −100° C. to 100° C.

(Eighth Process)

In this process, Wittig reaction is conducted to 4-position carbonyl group in 4-oxo-decahydronaphtho[2,3-c]furan derivatives represented by said general formula (1-2e), obtainable in said seventh process, by reacting ylide prepared from methyltriphenyl phosphonium salt and base, to prepare 4-methylene-dodecahydronaphtho[2,3-c]furan derivatives represented by said general formula (1-2f).

For the phosphonium salt to be used in this process, methyltriphenyl phosphonium chloride, methyltriphenyl phosphonium bromide or methyltriphenyl phosphonium iodide is mentioned and, for the base to be used, for example, alkali metal alkoxide such as sodium methoxide or sodium ethoxide, alkali metal organic base such as n-butyllithium, lithium bis(trimethylsilyl)amide, sodium bis(trimethylsilyl)amide or potassium bis(trimethylsilyl)amide, tertiary organic base such as triethylamine, diisopropylethylamine, pyridine, N-methylmorpholine, imidazole, pyrrolidine, piperidine, 1,5-diazabicyclo[4.3.0]non-5-ene or 1,8-diazabicyclo[5.4.0]undec-7-ene, or inorganic base such as potassium carbonate or sodium hydrogencarbonate can be present. For the solvent, any can be used, if it does not take part in the reaction, but, for example, hydrocarbonic solvent such as pentane, hexane, cyclohexane, benzene, toluene or xylene, halogenated hydrocarbonic solvent such as dichloromethane, 1,2-dichloroethane, chloroform or carbon tetrachloride, ethereal solvent such as diethyl ether, tetrahydrofuran or 1,4-dioxane, or alcoholic solvent such as methanol, ethanol, 1-propanol, 2-propanol, 1-butanol, 2-butanol, 2-methyl-1-propanol or 2-methyl-2-propanol is used suitably. The reaction proceeds smoothly at −110° C. to 100° C.

(Ninth Process)

In this process, the hydroboration-oxidation reaction is conducted to 4-position methylene group in 4-methylene-dodecahydronaphtho[2,3-c]furan derivatives represented by said general formula (1-2f), obtainable in said sixth process, to prepare 4-hydroxymethyl-dodecahydronaphtho[2,3-c]furan derivatives represented by said general formula (1-2g).

For the hydroborating agent to be used in this reaction, for example, borane-tetrahydrofuran complex, borane-dimethyl sulfide complex, 9-borabicyclo[3.3.1]nonane or the like is mentioned and, for the solvent, any can be used, if it does not take part in the reaction, but, for example, hydrocarbonic solvent such as pentane, hexane, cyclohexane, benzene, toluene or xylene, halogenated hydrocarbonic solvent such as dichloromethane, 1,2-dichloroethane, chloroform or carbon tetrachloride, ethereal solvent such as diethyl ether, tetrahydrofuran or 1,4-dioxane, or mixed solvent thereof is used. The reaction proceeds smoothly at −110° C. to 200° C. For the oxidation reaction to be conducted next, aqueous hydrogen peroxide, m-chloroperbenzoic acid, peracetic acid or the like is used. For the solvent, any can be used, if it does not take part in the reaction, but, for example, hydrocarbonic solvent such as pentane, hexane, cyclohexane, benzene, toluene or xylene, halogenated hydrocarbonic solvent such as dichloromethane, 1,2-dichloroethane, chloroform or carbon tetrachloride, ethereal solvent such as diethyl ether, tetrahydrofuran or 1,4-dioxane, alcoholic solvent such as methanol, ethanol, 1-propanol, 2-propanol, 1-butanol, 2-butanol, 2-methyl-1-propanol or 2-methyl-2-propanol, or mixed solvent thereof with water is used suitably. The reaction proceeds smoothly at −110° C. to 100° C. Besides, with several kinds in the compounds in this process, it is possible to separate optical isomers by means of optical resolution that utilizes asymmetric auxiliary or the like, HPLC technique or the like. In addition, it is also possible to separate stereoisomers in relation to 4-hydroxymethyl group under this separating condition.

(Tenth Process)

In this process, a lower acyl group, substituted or unsubstituted arylacyl group or elimination group is introduced to primary hydroxyl group of 4-hydroxymethyl-dodecahydronaphtho[2,3-c]furan derivatives represented by said general formula (1-2g), obtainable in said ninth process, to prepare dodecahydronaphtho[2,3-c]furan derivatives represented by said general formula (1-2h) (R₂₅ denotes a lower alkyl group, substituted or unsubstituted aralkyl group, lower acyl group, arylacyl group which may have one or more substituents, or elimination group united with oxygen atom).

For the reacting agent to be usable in this reaction, for example, benzoyl chloride, 4-bromobenzoyl chloride, methanesulfonyl chloride, p-toluenesulfonyl chloride or the like is mentioned. For the solvent, any can be used, if it does not take part in the reaction, but, for example, hydrocarbonic solvent such as pentane, hexane, cyclohexane, benzene, toluene or xylene, halogenated hydrocarbonic solvent such as dichloromethane, 1,2-dichloroethane, chloroform or carbon tetrachloride, ethereal solvent such as diethyl ether, tetrahydrofuran or 1,4-dioxane, alcoholic solvent such as methanol, ethanol, 1-propanol, 2-propanol, 1-butanol, 2-butanol, 2-methyl-1-propanol or 2-methyl-2-propanol, or mixed solvent thereof is used. For the base to be used, for example, alkali metal alkoxide such as sodium methoxide or sodium ethoxide, alkali metal organic base such as n-butyllithium, lithium bis(trimethylsilyl)amide, sodium bis(trimethylsilyl)amide or potassium bis(trimethylsilyl) amide, tertiary organic base such as triethylamine, diisopropylethylamine, pyridine, N-methylmorpholine, imidazole, pyrrolidine, piperidine, 1,5-diazabicyclo[4.3.0]non-5-ene or 1,8-diazabicyclo[5.4.0]undec-7-ene, or inorganic base such as potassium carbonate or sodium hydrogencarbonate can be present or absent. The reaction proceeds smoothly at −110° C. to 200° C.

(Eleventh Process)

In this process, sulfuric nucleophilic reacting agent such as thiophenol is reacted to 4-position hydroxymethyl group substituted with elimination group in dodecahydronaphtho[2,3-c]furan derivatives represented by said general formula (1-2h), obtainable in said tenth process, to prepare 4-arylsulfenylmethyl-dodecahydronaphtho[2,3-c]furan derivatives represented by said general formula (1-2i).

For the reacting agent to be usable in this reaction, for example, said thiophenol, p-toluenethiophenol or the like is mentioned. For the solvent, any can be used, if it does not take part in the reaction, but, for example, hydrocarbonic solvent such as pentane, hexane, cyclohexane, benzene, toluene or xylene, halogenated hydrocarbonic solvent such as dichloromethane, 1,2-dichloroethane, chloroform or carbon tetrachloride, ethereal solvent such as diethyl ether, tetrahydrofuran or 1,4-dioxane, alcoholic solvent such as methanol, ethanol, 1-propanol, 2-propanol, 1-butanol, 2-butanol, 2-methyl-1-propanol or 2-methyl-2-propanol, or mixed solvent thereof is used. For the base to be used, for example, alkali metal alkoxide such as sodium methoxide or sodium ethoxide, alkali metal organic base such as n-butyllithium, lithium bis(trimethylsilyl)amide, sodium bis(trimethylsilyl)amide or potassium bis(trimethylsilyl)amide, tertiary organic base such as triethylamine, diisopropylethylamine, pyridine, N-methylmorpholine, imidazole, pyrrolidine, piperidine, 1,5-diazabicyclo[4.3.0]non-5-eneor 1,8-diazabicyclo[5.4.0]undec-7-ene, or inorganic base such as potassium carbonate or sodium hydrogencarbonate can be present or absent. The reaction proceeds smoothly at −110° C. to 200° C.

(Twelfth Process)

In this process, oxidation reaction is conducted to sulfenyl group in 4-arylsulfenylmethyl-dodecahydronaphtho[2,3-c]furan derivatives represented by said general formula (1-2i), obtainable in said eleventh process, to prepare 4-arylsulfonylmethyl-dodecahydronaphtho[2,3-c]furan derivatives represented by said general formula (1-2j).

For the reacting agent to be used in this process, for example, aqueous hydrogen peroxide, m-chloroperbenzoic acid, peracetic acid or the like is used. For the solvent, any can be used, if it does not take part in the reaction, but, for example, hydrocarbonic solvent such as pentane, hexane, cyclohexane, benzene, toluene or xylene, halogenated hydrocarbonic solvent such as dichloromethane, 1,2-dichloroethane, chloroform or carbon tetrachloride, ethereal solvent such as diethyl ether, tetrahydrofuran or 1,4-dioxane, alcoholic solvent such as methanol, ethanol, 1-propanol, 2-propanol, 1-butanol, 2-butanol, 2-methyl-1-propanol or 2-methyl-2-propanol, or mixed solvent thereof with water is used suitably. For the base, for example, alkali metal alkoxide such as sodium methoxide or sodium ethoxide, alkali metal organic base such as n-butyllithium, lithium bis(trimethylsilyl)amide, sodium bis(trimethylsilyl)amide or potassium bis(trimethylsilyl)amide, tertiary organic base such as triethylamine, diisopropylethylamine, pyridine, N-methylmorpholine, imidazole, pyrrolidine, piperidine, 1,5-diazabicyclo[4.3.0]non-5-ene or 1,8-diazabicyclo[5.4.0]undec-7-ene, or inorganic base such as potassium carbonate or sodium hydrogencarbonate can be present or absent. The reaction proceeds smoothly at −110° C. to 200° C.

(Thirteenth Process)

In this process, piperidine derivatives represented by said general formula (6) is coupled to 4-arylsulfonylmethyl-dodecahydronaphtho[2,3-c]furan derivatives represented by said general formula (1-2j), obtainable in said twelfth process and P-hydroxysulfone derivatives produced is treated reductively, thereby preparing dodecahydronaphtho[2,3-c]furan derivatives represented by said general formula (1-1a). For the solvent, any can be used, if it does not take part in the reaction, but, for example, hydrocarbonic solvent such as pentane, hexane, cyclohexane, benzene, toluene or xylene, halogenated hydrocarbonic solvent such as dichloromethane, 1,2-dichloroethane, chloroform or carbon tetrachloride, ethereal solvent such as diethyl ether, tetrahydrofuran or 1,4-dioxane, alcoholic solvent such as methanol, ethanol, 1-propanol, 2-propanol, 1-butanol, 2-butanol, 2-methyl-1-propanol or 2-methyl-2-propanol, or mixed solvent thereof with water is used suitably. For the base, for example, alkali metal alkoxide such as sodium methoxide or sodium ethoxide, alkali metal organic base such as n-butyllithium, lithium bis(trimethylsilyl) amide, sodium bis(trimethylsilyl)amide or potassium bis(trimethylsilyl)amide, tertiary organic base such as triethylamine, diisopropylethylamine, pyridine, N-methylmorpholine, imidazole, pyrrolidine, piperidine, 1,5-diazabicyclo[4.3.0]non-5-ene or 1,8-diazabicyclo[5.4.0]undec-7-ene, or inorganic base such as potassium carbonate or sodium hydrogencarbonate can be present or absent. The reaction proceeds smoothly at −110° C. to 200° C.

In the reductive treatment to be conducted next, sodium amalgam, samarium iodide or the like is used. Or, β-hydroxysulfone may be once converted to acetoxy or benzoyl and, after this is submitted to β-elimination using a base, sulfonyl group may be removed reductively. For the solvent, any can be used, if it does not take part in the reaction, but, for example, hydrocarbonic solvent such as pentane, hexane, cyclohexane, benzene, toluene or xylene, halogenated hydrocarbonic solvent such as dichloromethane, 1,2-dichloroethane, chloroform or carbon tetrachloride, ethereal solvent such as diethyl ether, tetrahydrofuran or 1,4-dioxane, alcoholic solvent such as methanol, ethanol, 1-propanol, 2-propanol, 1-butanol, 2-butanol, 2-methyl-1-propanol or 2-methyl-2-propanol, or mixed solvent thereof with water is used suitably. For the base, for example, alkali metal alkoxide such as sodium methoxide or sodium ethoxide, alkali metal organic base such as n-butyllithium, lithium bis(trimethylsilyl)amide, sodium bis(trimethylsilyl)amide or potassium bis(trimethylsilyl)amide, tertiary organic base such as triethylamine, diisopropylethylamine, pyridine, N-methylmorpholine, imidazole, pyrrolidine, piperidine, 1,5-diazabicyclo[4.3.0]non-5-ene or 1,8-diazabicyclo[5.4.0]undec-7-ene, or inorganic base such as potassium carbonate or sodium hydrogencarbonate can be present or absent. The reaction proceeds smoothly at −110° C. to 200° C.

(Fourteenth Process)

In this process, 1-position protective group in dodecahydronaphtho[2,3-c]furan derivatives represented by said general formula (1-1a), obtainable in said thirteenth process, is removed oxidatively to prepare 1-oxodecahydronaphtho[2,3-c]furan derivatives represented by said general formula (1-1b).

For the reacting agent to be used in this process, oxidizer such as Jones reagent is used usually. For the solvent, any can be used, if it does not take part in the reaction, but, for example, halogenated hydrocarbonic solvent such as dichloromethane, 1,2-dichloroethane, chloroform or carbon tetrachloride is used. The reaction proceeds smoothly at −100° C. to 100° C. Or, compounds represented by said general formula (1-1a) (R₉ denotes a lower alkyl group, substituted or unsubstituted aralkyl group, lower acyl group, lower alkoxy group, substituted or unsubstituted aralkyloxy group or lower acyloxy group) may be reacted in acidic aqueous solution to prepare compounds represented by said general formula (1-1a) (R₉ denotes a hydroxyl group) and then compounds represented by said general formula (1-1b) may be prepared by reacting under usual condition of oxidation reaction. For the “acidic aqueous solution” to be used in this reaction, aqueous solution of inorganic acid such as hydrochloric acid, sulfuric acid, nitric acid or phosphoric acid, organic acid such as acetic acid, citric acid, succinic acid, fumaric acid, maleic acid or tartaric acid is mentioned. Moreover, the oxidation reaction can be conducted by using chromic acid, chromium trioxide-pyridine mixed system, dimethyl sulfoxide-oxalyl chloride-triethylamine mixed system, ruthenium complex, Dess-Martin reagent or the like. This process is usually desirable to be performed in a solvent and, for example, halogenated hydrocarbonic solvent such as dichloromethane, 1,2-dichloroethane, chloroform or carbon tetrachloride is used. The reaction proceeds smoothly at −100° C. to 100° C.

Furthermore, in the case of 1-oxodecahydronaphtho[2,3-c]furan derivatives represented by said general formula (1-1b) (R₆ denotes a protective group of amino group), 1-oxodecahydronaphtho[2,3-c]furan derivatives represented by said general formula (1-1b) (R₆ denotes a hydrogen atom) can be prepared by removing this protective group, or 1-oxodecahydronaphtho[2,3-c]furan derivatives represented by said general formula (1-1b) (R₆ denotes a lower alkyl group or substituted or unsubstituted aralkyl group) can be prepared by further reductively alkylating these compounds.

The removal of the protective group of amino group can be performed by appropriately adopting the method described in the reference (Green, T. W.; Wuts, P. G. M. “Protective Groups in organic Synthesis”, 2^{nd} Ed., Wiley Interscience Publication, John-Wiley & Sons, New York, 1991, pp 14-118). For the solvent, any can be used, if it does not take part in the reaction, but, for example, hydrocarbonic solvent such as pentane, hexane, cyclohexane, benzene, toluene or xylene, halogenated hydrocarbonic solvent such as dichloromethane, 1,2-dichloroethane, chloroform or carbon tetrachloride, ethereal solvent such as diethyl ether, tetrahydrofuran or 1,4-dioxane, alcoholic solvent such as methanol, ethanol, 1-propanol, 2-propanol, 1-butanol, 2-butanol, 2-methyl-1-propanol or 2-methyl-2-propanol, or mixed solvent thereof with water is used suitably. The reaction proceeds smoothly at −110° C. to 200° C. The reductive alkylation to be conducted next can be performed by reacting a reducing agent such as sodium cyanoborohydride or sodium borohydride in the presence of corresponding alkyl aldehyde. For the solvent, any can be used, if it does not take part in the reaction, but, for example, hydrocarbonic solvent such as pentane, hexane, cyclohexane, benzene, toluene or xylene, halogenated hydrocarbonic solvent such as dichloromethane, 1,2-dichloroethane, chloroform or carbon tetrachloride, ethereal solvent such as diethyl ether, tetrahydrofuran or 1,4-dioxane, alcoholic solvent such as methanol, ethanol, 1-propanol, 2-propanol, 1-butanol, 2-butanol, 2-methyl-1-propanol or 2-methyl-2-propanol, or mixed solvent thereof with water is used suitably. The reaction proceeds smoothly at −110° C. to 200° C.

### BEST EMBODIMENT TO PUT THE INVENTION INTO PRACTICE

In following, the invention will be illustrated in detail based on examples and referential examples, but it goes without saying that the invention is not confined thereto.

### EXAMPLE 1

3a,4,5,6,7,8,9,9a-Octahydro-4,9-epoxynaphtho[2,3-c]furan-1(3H)-one

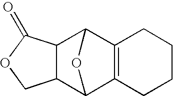

To 3.00 g (35.7 mmol) of furan-2(5H)-one were added 0.96 g (1.78 mmol) of 4,4′-thiobis(6-tert-butyl-m-cresol), 6 mL of a solution of 6.54 g (53.5 mmol) of 4,5,6,7-tetrahydroisobenzofuran in dehydrated ether and 3.18 g of lithium perchlorate, and, after argon flushing, the mixture was stirred for 48 hours at room temperature. The reaction mixture was diluted with 100 mL of ether and poured into 100 ml of water, followed by separation of two layers. The aqueous layer was extracted with methylene chloride (10 ml×3). Combined organic layers were dried over anhydrous magnesium sulfate, filtered and solvent was distilled off under reduced pressure. The residue was purified by means of silica gel column chromatography (hexane:ethyl acetate=4:1, then 2:1, and then 1:1) to obtain 3.62 g of title compound (yield 49%). mp. 105-106° C.

¹H-NMR(400 MHz, CDCl₃): δ1.46-1.56(m, 2H), 1.63-1.72(m, 2H), 1.84-1.97(m, 2H), 2.19-2.30(m, 2H), 2.72(td, J=8.3, 3.6 Hz, 1H), 2.81(d, J=7.8 Hz, 1H), 4.21(dd, J=9.8, 3.9 Hz, 1H), 4.51(dd, J=9.8, 8.6 Hz, 1H), 4.71(s, 1H), 5.04(s, 1H). IR(KBr); 2950, 1750, 1190 cm⁻¹. HRMS(EI)(m/z): Calcd. for C₁₂H₁₄O₃ (M⁺): 206.0943. Found, 206.0970.

### EXAMPLE 2

Decahydro-4,9-epoxynaphtho[2,3-c]furan-1(3H)-one

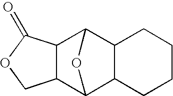

To 300 mL of a solution of 17.0 g (82.4 mmol) of the compound of Example 1 in ethanol were added 1.70 g (10% by weight) of 10% palladium-carbon, and the mixture was submitted to catalytic reduction at ambient temperature and at 98.1 KPa. After stirring for 5 hours, the reaction mixture was filtered through celite and the residue was washed using 100 mL of ethyl acetate. Combined organic layers were distilled off under reduced pressure to obtain 16.8 g of title compound (yield 98%).

mp. 90-91° C. (hexane-ethyl acetate).

¹H-NMR(400 MHz, CDCl₃): δ1.04-1.21(m, 2H), 1.30-1.57(m, 4H), 1.68-1.78(m, 2H), 2.01-2.17(m, 2H), 3.00(td, J=8.3, 3.9 Hz, 1H), 3.05(d, J=8.3 Hz, 1H), 4.10(dd, J=9.3, 4.0 Hz, 1H), 4.40(d, J=4.9 Hz, 1H), 4.46(t, J=9.1 Hz, 1H), 4,75(d, J=4.9 Hz, 1H). ¹³C-NMR(100 MHz, CDCl₃): δ19.4, 19.4, 19.5, 19.8, 37.8, 38.9, 39.6, 45.0, 72.4, 83.7, 86.2, 178.6. IR(KBr); 2940, 1760 cm⁻¹. Anal. for C₁₂H₁₆O₃: calcd., C: 69.21, H: 7.74; found, C: 68.97, H: 7.74.

### EXAMPLE 3

3a,4,4a,5,6,7,8,8a-octahydro-4-hydroxynaphtho[2,3-c]furan-1(3H)-one

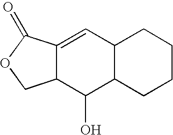

To 500 mL of a solution of 10.0 g (48.0 mmol) of the compound of Example 2 in dehydrated tetrahydrofuran were added dropwise 228.7 mL (0.24 mol) of lithium bis(trimethylsilyl)amide-hexane solution (1.05 mol/L) at −70° C., and the mixture was stirred for about 5 hours while naturally raising the temperature to −40° C. To the reaction mixture were added 500 mL of cold dilute aqueous solution of citric acid. Solvent was distilled off and the residue was extracted with ether (150 ml×3). The organic layer was washed with brine (100 mL), dried over anhydrous magnesium sulfate, filtered and solvent was distilled off. The residue was purified by means of silica gel column chromatography (hexane:ethyl acetate=1:1) to obtain 9.87 g of title compound (yield 99%).

¹H-NMR(400 MHz, CDCl₃): δ0.98-1.74(m, 4H), 1.81-1.89(m, 2H), 1.95-2.10(m, 2H), 2.14-2.21(m, 1H), 2.31-2.38(m, 1H), 2.77-2.84(m, 1H), 3.16-3.23(m, 1H), 4.03(dd, J=9.3, 3.4 Hz, 1H), 4.31(dd, J=9.8, 8.3 Hz, 1H), 4.45(dd, J=8.8, 8.1 Hz, 1H), 6.75(t, J=2.8 Hz, 1H). HRMS(EI)(m/z): Calcd. forC₁₂H₁₆O₃ (M+): 208.1099. Found, 208.1086.

### EXAMPLE 4

3a,4,4a,5,6,7,8,9a-Octahydro-4-hydroxynaphtho[2,3-c]furan-1(3H)-one

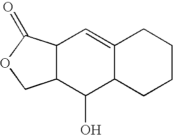

To 5 mL of a solution of 1.89 g (9.08 mmol) of the compound of Example 3 in dehydrated toluene were added 6.79 mL (45.4 mmol) of 1,8-diazabicyclo[5.4.0]undec-7-ene, and the mixture was stirred for 4 hours at about 80° C. After cooling, solvent was distilled off. The residue was diluted with 50 mL of ether and 50 mL of cold dilute aqueous solution of citric acid were added for washing. The aqueous layer was extracted with ether (10 ml×3). Combined organic layers were washed with brine (20 mL), dried over anhydrous magnesium sulfate, filtered and solvent was distilled off. The residue was purified by means of silica gel column chromatography (hexane:ethyl acetate=1:2) to obtain 1.33 g of title compound (yield 70%).

¹H-NMR(400 MHz, CDCl₃): δ1.03(qd, J=12.7, 3.5 Hz, 1H), 1.19-1.33(m, 1H), 1.38-1.49(m, 1H), 1.73(d, J=3.9 Hz, 1H), 1.79-1.90(m, 2H), 1.96-2.10(m, 2H), 2.14-2.22(m, 1H), 2.31-2.38(m, 1H), 3.02(qd, J=8.3, 4.4 Hz, 1H), 3.21(dq,J=8.3, 2.8 Hz, 1H), 3.83(dt, J=7.3, 4.4 Hz, 1H), 4.24(t, J=8.8 Hz, 1H), 4.45(dd, J=9.3, 8.1 Hz, 1H), 5.34(d, J=3.0 Hz, 1H). ¹³C-NMR(100 MHz, CDCl₃): δ25.7, 27.1, 31.8, 34.8, 39.0, 41.3, 41.5, 68.6, 72.0, 111.9, 141.5, 177.0.

IR(neat); 3450, 2930, 1770, 1010 cm⁻¹. HRMS(EI)(m/z): Calcd. for C₁₂H₁₆O₃ (M+): 208.1099. Found, 208.1095.

### EXAMPLE 5

Decahydro-4-hydoxy-naphtho[2,3-c]furan-1(3H)-one

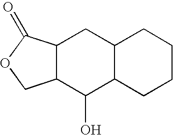

To 100 mL of a solution of 5.12 g (24.6 mmol) of the compound of Example 4 in ethanol were added 0.50 g (10% by weight) of platinum oxide, and the mixture was submitted to catalytic reduction at ambient temperature and at 98.1 KPa. After stirring for 2 hours, the reaction mixture was filtered through celite and the residue was washed using 100 mL of ethyl acetate. Combined organic layers were distilled off under reduced pressure to obtain 4.93 g of title compound (yield 95%).

mp. 149-150° C. (hexane-ethyl acetate).

¹H-NMR(400 MHz, CDCl₃): δ0.83-0.93(m, 1H), 0.98-1.32(m, 6H), 1.69-1.86(m, 4H), 1.96(d, J=3.9 Hz, 1H), 2.05-2.12(m, 1H), 2.62(dt, J=12.7, 6.9 Hz, 1H), 3.01(dq, J=11.7, 7.2 Hz, 1H), 3.66(ddd, J=10.3, 6.4, 3.7 Hz, 1H), 4.31(dd, J=11.3, 9.3 Hz, 1H), 4.43(dd, J=9.3, 8.3 Hz, 1H). ¹³C-NMR(100 MHz, CDCl₃): δ25.6, 25.6, 28.9, 30.6, 32.9, 38.2, 39.6, 40.8, 43.6, 68.1, 73.0, 178.9. IR(KBr); 3440, 2920, 1750, 1190 cm⁻¹. Anal. for C₁₂H₁₈O₃: calcd., C: 68.55, H: 8.63; found, C: 68.52, H: 8.81.

### EXAMPLE 6

Dodecahydro-1-methoxy-naphtho[2,3-c]furan-4-ol

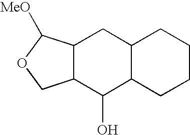

To 500 mL of a solution of 8.00 g (38.0 mmol) of the compound of Example 5 in dehydrated ether were added dropwise 122.7 mL (0.11 mol) of diisobutyl aluminum hydride-n-hexane solution (0.93 mol/L) at −70° C. under an atmosphere of argon. After stirring for about 1 hour at the same temperature, methanol-water mixed solution (10 mL+10 mL) was added dropwise slowly and the mixture was stirred for 1 hour at room temperature. The residue was filtered through celite and washed with ethyl acetate-methanol mixed solution (10:1) (300 mL). Solvent was distilled off and 300 mL of brine were added, which was extracted with methylene chloride-ethanol (10:1) mixed solution (50 mL×5). The solution was dried over anhydrous magnesium sulfate, filtered and solvent was distilled off to obtain 6.88 g of crude hemiacetal form being colorless oily product (yield 85%).

¹H-NMR(400 MHz, CDCl₃): δ0.83-1.30(m, 7H), 1.43-1.85(m, 5H), 2.02-2.09(m, 1H), 2.21(dt, J=12.2, 6.2 Hz, 1H), 2.46(d, J=2.9 Hz, 1H), 3.03-3.11(m, 1H), 3.68(ddd, J=9.8, 5.9, 3.7 Hz, 1H), 3.98(dd, J=10.3, 8.8 Hz, 1H), 4.16(apparent t, J=8.8 Hz, 1H), 5.15(d, J=3.0 Hz, 1H). HRMS(CI)(m/z): Calcd. for C₁₂H₁₉O₂ (MH⁺—H₂O): 195.1385. Found, 195.1364.

This was dissolved into a mixed solution of 200 mL of dehydrated methanol and 200 mL of dehydrated methylene chloride, 5.98 mL (48.6 mmol) of boron trifluoride-diethyl ether complex were added dropwise at −70° C. under an atmosphere of argon, and the mixture was stirred for about 12 hours while naturally raising the temperature. To the reaction mixture were added 6.78 mL (48.6 mmol) of triethylamine, and, after stirring at room temperature, solvent was distilled off. To the residue were added 300 mL of cold dilute aqueous solution of citric acid, which was extracted with ether (100 ml×3). The organic layer was washed with brine (100 mL), dried over anhydrous magnesium sulfate, filtered and solvent was distilled off. The residue was purified by means of silica gel column chromatography (hexane:ethyl acetate=2:1) to obtain 4.56 g of title compound (yield 62%). mp. 75-77° C. (hexane-ethyl acetate).

¹H-NMR(400 MHz, CDCl₃): δ0.81-1.05(m, 4H), 1.11-1.30(m, 3H), 1.45-1.51(m, 1H), 1.63(d, J=3.9 Hz, 1H), 1.63-1.84(m, 3H), 2.02-2.09(m, 1H), 2.17(dt, J=12.2, 6.2 Hz, 1H), 2.93-3.01(m, 1H), 3.32(s, 3H), 3.65(ddd, J=10.3, 6.4, 4.2 Hz, 1H), 3.97(dd, J=10.3, 8.3 Hz, 1H), 4.06(t, J=9.1 Hz, 1H), 4.63(s, 1H). ¹³C-NMR(100 MHZ, CDCl₃): δ25.9, 25.9, 29.0, 32.0, 33.3, 38.4, 41.4, 43.3, 44.2, 54.4, 67.8, 74.5, 109.5. IR(KBr); 3350, 2920, 1040 cm⁻¹. Anal. for C₁₃H₂₂O₃: calcd., C: 68.99, H: 9.80; found, C: 68.70, H: 9.66.

### EXAMPLE 7

Decahydro-1-methoxy-naphtho[2,3-c]furan-4(1H)-one

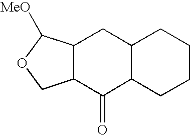

To 6 mL of a solution of 691.4 mg (3.06 mmol) of the compound of Example 6 in methylene chloride were added 536.9 mg (4.58 mmol) of 4-methylmorpholine N-oxide and 1.50 g of molecular sieves (MS) 4A, then, after 53.7 mg (0.15 mmol) of tetrapropylammonium perruthenate (TPAP) were added, the mixture was stirred for 1 hour at room temperature. The reaction mixture was filtered through celite, the residue was washed with ether (30 mL), and solvent was distilled off. The residue was diluted with 50 mL of ether and washed with aqueous solution of 10% Na₂S₂O₃, then with brine (each 20 mL). The organic layer was dried over anhydrous magnesium sulfate, filtered and solvent was distilled off. The residue was purified by means of silica gel column chromatography (hexane: ethyl acetate=2:1) to obtain 667.0 mg of title compound (yield 97%).

mp. 78-79° C. (hexane).

¹H-NMR(400 MHz, CDCl₃): δ1.08-1.33(m, 5H), 1.39-1.49(m, 1H), 1.68-1.86(m, 4H), 1.95-2.04(m, 2H), 2.56(dt, J=12.7, 6.6 Hz, 1H), 3.21-3.31(m, 1H), 3.31(s, 3H), 3.95(t, J=9.1 Hz, 1H), 4.17(dd, J=10.3, 8.8 Hz, 1H), 4.77(s, 1H). ¹³C-NMR(100 MHz, CDCl₃): δ25.1, 25.4, 25.5, 32.5, 34.2, 40.9, 48.0, 50.0, 50.8, 54.4, 69.1, 109.4, 211.4.

IR(KBr); 2940, 1700 cm⁻¹. Anal. for C₁₃H₂₀O₃: calcd., C: 69.61, H: 8.99; found, C: 69.32, H: 9.07.

### EXAMPLE 8

Dodecahydro-1-methoxy-4-methylenenaphtho[2,3-c]furan

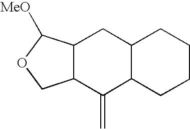

To 200 mL of a suspension of 13.2 g (32.5 mmol) of methyltriphenylphosphonium iodide in dehydrated ether were added dropwise 32.5 mL (32.5 mmol) of sodium bis(trimethylsilyl)amide-toluene solution (1 mol/L) at 0° C. under an atmosphere of argon. After stirring for 30 minutes at room temperature, 30 mL of a solution of 1.46 g (6.51 mmol) of the compound of Example 7 in dehydrated ether were added dropwise at −78° C. After stirring for about 6 hours while naturally raising the temperature, 100 mL of cold saturated aqueous solution of ammonium chloride were added to the reaction mixture. The solution was filtered through celite, the residue was washed with ether (200 mL), and solvent was distilled off. To the residue were poured 50 mL of water, which was extracted with ether (30 ml×3). The organic layer was washed with brine (30 mL), dried over anhydrous magnesium sulfate, filtered and solvent was distilled off. The residue was purified by means of silica gel column chromatography (hexane: ethyl acetate=10:1) to obtain 777.6 mg of title compound (yield 54%).

mp. 53-55° C.

¹H-NMR(400 MHz, CDCl₃): δ1.00-1.35(m, 6H), 1.54-1.73(m, 4H), 1.80-1.89(m, 2H), 2.17-2.24(m, 1H), 3.26-3.32(m, 1H), 3.33(s, 3H), 3.79(dd, J=10.3, 8.1 Hz, 1H), 4.00(dd, J=9.3, 8.3 Hz, 1H), 4.64(s, 1H) 4.72(t, J=2.0 Hz, 1H), 4.86(t, J=1.8 Hz, 1H). ¹³C-NMR(100 MHz, CDCl₃): δ26.1, 26.3, 28.8, 32.9, 34.4, 41.4, 42.0, 45.1, 46.5, 54.5, 70.6, 109.3, 109.9, 148.5. IR(KBr); 2930, 1440, 1380, 1190, 1030 cm⁻¹. HRMS(EI)(m/z): Calcd. for C₁₄H₂₂O₂ (M⁺): 222.1620. Found, 222.1605.

### EXAMPLE 9

Dodecahydro-1-methoxy-naphtho[2,3-c]furan-4-methanol

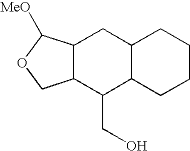

To 40 mL of a solution of 880.0 mg (3.96 mmol) of the compound of Example 8 in dehydrated tetrahydrofuran were added dropwise 5.94 mL (5.94 mmol) of borane-tetrahydrofuran complex (1 mol /L) at −78° C. under an atmosphere of argon, and the mixture was stirred for 18 hours while naturally raising the temperature to 0° C. To the reaction mixture were added 10 mL of water to stop the reaction, and then 4.49 mL of 30% aqueous hydrogen peroxide and 4.49 mL of 10% aqueous solution of sodium hydroxide were added, which was stirred for 1.5 hours. Solvent was distilled off and 20 mL of water were poured to the residue, which was extracted with ether (5 ml×3). The organic layer was washed with brine (10 mL), dried over anhydrous magnesium sulfate, filtered and solvent was distilled off under reduced pressure. The residue was purified by means of silica gel column chromatography (hexane:ethyl acetate=1:1) to obtain 321.2 mg of title compound being a mixture of four kinds of isomers (yield 34%).

Rf=0.45 (hexane:ethyl acetate=1:1).

HRMS(CI)(m/z):Calcd. forC₁₄H₂₅O₃ (MH⁺): 241.1804. Found, 241.1787.

### EXAMPLE 10

Dodecahydro-4-(4-bromophenyl)carboxymethyl-1-methoxynaphtho[2,3-c]furan

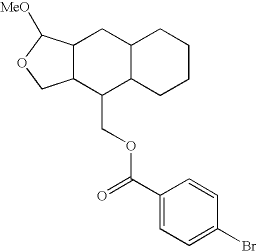

To 2 mL of a solution of 225.8 mg (1.12 mmol) of 4-bromobenzoic acid in benzene were added 0.50 mL of thionyl chloride, and the mixture was stirred for 1 hour at 80° C. Solvent was distilled off, the residue was diluted with 2 mL of methylene chloride, and 90.0 mg (0.37 mmol) of the compound of Example 9 were added, then 261.0 μL (1.87 mmol) of triethylamine were added dropwise. The mixture was stirred for about 18 hours while naturally raising the temperature. Solvent was distilled off and 10 mL of dilute aqueous solution of citric acid were poured into the residue, which was extracted with ethyl acetate (3 ml×3). The organic layer was washed with brine (3 mL), dried over anhydrous magnesium sulfate, filtered and solvent was distilled off under reduced pressure. The residue was purified by means of silica gel column chromatography (hexane:ethyl acetate=4:1) to obtain 157.0 mg of title compound being a mixture of four kinds of isomers (yield 99%).

Rf=0.30 (hexane:ethyl acetate=4:1).

HRMS(FAB) (m/z): Calcd. for C₂₅H₃₈BrO₆S₂ [(M+2,2′-dithiodiethanol) H⁺]: 577.1293. Found, 577.1246.

Said mixture was submitted to the optical resolution with CHIRALCEL OD (from Daicel Chemical Industries, Ltd.) (developing solvent:hexane:2-propanol=50:1), and isomers A, B, C and D were obtained in order of higher elution speed.

Isomer A: [α]_{D}²⁶+5.7° (c 0.10, CHCl₃). mp. 107-108° C. (hexane), Colorless prismatic crystals.

¹H-NMR(400 MHz, CDCl₃): δ0.82-0.96(m, 2H), 1.16-1.43(m, 6H), 1.53-1.73(m, 3H), 1.77-1.82(m, 1H), 1.94-1.99(m, 1H), 2.23(dt, J=12.7, 6.4 Hz, 1H), 2.83(dt, J=10.1, 5.9 Hz, 1H), 3.31(s, 3H), 3.83(dd, J=11.3, 8.1 Hz, 1H), 4.07(dd, J=9.3, 7.8 Hz, 1H), 4.26(dd, J=11.3, 8.3 Hz, 1H), 4.50(dd, J=10.8, 4.2 Hz, 1H), 4.61(s, 1H), 7.59(ddd, J=8.8, 2.3, 2.3 Hz, 2H), 7.89(ddd, J=8.8, 2.3, 2.3 Hz, 2H). ¹³C-NMR(100 MHz, CDCl₃): δ26.2, 27.1, 30.4, 32.8, 34.7, 34.8, 36.4, 37.7, 39.1, 41.1, 54.5, 66.4, 69.5, 110.2, 128.0, 129.2, 131.1, 131.1, 131.8, 131.8, 166.0. MS(FAB)m/z: 528 [(M+diethanolamine)H⁺]. IR(KBr); 2940, 1710, 1590, 1270, 1110 cm⁻¹. HRMS(FAB)(m/z): Calcd. for C₂₅H₃₉BrNO₆ [(M+diethanolamine)H⁺]: 528.1961. Found, 528.1935. Anal. for C₂₁H₂₇BrO₄: calcd., C: 59.58, H: 6.43; found, C: 59.47, H: 6.32.

Isomer B: [α]_{D}²²−41° (c 0.63, CHCl₃). mp. 94-95° C. (pentane-ether), Colorless prismatic crystals.

¹H-NMR(400 MHz, CDCl₃): δ0.69-1.34(m, 6H), 1.52-1.81(m, 6H), 1.85-1.98(m, 2H), 2.17(dt, J=11.7, 5.9 Hz, 1H), 2.82-2.89(m, 1H), 3.32(s, 3H), 3.94(dd, J=10.8, 8.3 Hz, 1H), 4.00(apparentt, J=8.3 Hz, 1H), 4.22(dd, J=11.7, 7.1 Hz, 1H), 4.36(dd, J=11.7, 3.7 Hz, 1H), 4.59(s, 1H), 7.59(dt, J=8.8, 2.1 Hz, 2H), 7.87(dt, J=8.8, 2.1 Hz, 2H). ¹³C-NMR(100 MHZ, CDCl₃): δ26.0, 26.4, 29.7, 30.1, 32.1, 34.0, 38.3, 38.4, 40.6, 44.7, 54.5, 66.3, 67.7, 109.1, 128.2, 129.0, 131.1, 131.1, 131.8, 131.8, 165.8.

MS(FAB)m/z: 391 (M⁺—OMe). IR(KBr); 2920, 1720, 1590, 1270, 1100 cm⁻¹. HRMS(FAB)(m/z): Calcd. for C₂₀H₂₄BrO₃ (M⁺—OMe): 391.0909. Found, 391.0949. Anal. for C₂₁H₂₇BrO₄: calcd., C: 59.58, H: 6.43; found, C: 59.47, H: 6.30.

Isomer C: [α]_{D}²³+42° (c 0.16, CHCl₃). mp. 93-94° C. (pentane-ether), Colorless prismatic crystals.

HRMS(FAB)(m/z): Calcd. for C₂₀H₂₄BrO₃ (M⁺-OMe): 391.0909. Found, 391.0949. Anal. for C₂₁H₂₇BrO₄: calcd., C: 59.58, H: 6.43; found, C: 59.68, H: 6.37. ¹H-NMR, ¹³C-NMR, MS and IR spectra were consistent completely with those of isomer B.

Isomer D: [α]_{D}²⁶−5.5° (c 0.10, CHCl₃). mp. 108-109° C. (hexane), Colorless prismatic crystals.

HRMS(FAB)(m/z): Calcd. for C₂₅H₃₉BrNO₆ [(M+diethanolamine)H⁺]: 528.1961. Found, 528.1920. Anal. for C₂₁H₂₇BrO₄: calcd., C: 59.58, H: 6.43; found, C: 59.40, H: 6.26.

¹H-NMR, ¹³C-NMR, MS and IR spectra were consistent completely with those of isomer A.

### EXAMPLE 11

Dodecahydro-1-methoxy-4-[(phenylsulfonyl)methyl]naphtho[2,3-c]furan

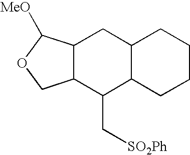

To 10 mL of a solution of 318.0 mg (1.32 mmol) of the compound of Example 9 in methylene chloride were added 922.1 μL (6.62 mmol) of triethylamine, and, after 307.2 μL (3.97 mmol) of methanesulfonyl chloride were added dropwise under cooling with ice and under an atmosphere of argon, the mixture was stirred for about 7 hours while naturally raising the temperature. Solvent was distilled off under reduced pressure and 20 mL of water were added to the residue, which was extracted with ether (5 ml×3). Combined organic layers were washed with brine (5 mL), dried over anhydrous magnesium sulfate, filtered and solvent was distilled off. The residue was purified by means of silica gel column chromatography (hexane:ethyl acetate=2:1) to obtain 374.1 mg of O-mesyl form (yield 89%). To 10 mL of a solution of 197.7 mg (1.76 mmol) of potassium t-butoxide in dehydrated dimethyl sulfoxide were added dropwise 181.0 μL (1.76 mmol) of thiophenol at room temperature under stirring, and the mixture was stirred for 10 minutes. This was added to 10 mL of a solution of 374.1 mg (1.17 mmol) of said O-mesyl form in dehydrated dimethyl sulfoxide and the mixture was stirred for about 24 hours at room temperature. The reaction mixture was poured into 20 mL of cold water, which was extracted with ether (10 ml×3). The aqueous layer was extracted (with chloroform:ethanol=10:1) (5 ml×3) and combined organic layers were dried over anhydrous magnesium sulfate, filtered and then solvent was distilled off to obtain crude phenyl thioether form. This was dissolved into 20 mL of methylenechloride and 724.1 mg (4.41 mmol) of m-chloroperbenzoic acid (70%) and 493.5 mg (4.41 mmol) of sodium hydrogencarbonate were added under cooling with ice and stirring. After stirring for 4 hours at room temperature, the reaction mixture was filtered through celite and the residue was washed with methylene chloride (50 ml). After solvent was distilled off under reduced pressure, saturated aqueous solution of sodium hydrogencarbonate (10 ml) was added, which was extracted with ether (3 ml×3). The organic layer was washed with brine (3 ml), dried over anhydrous magnesium sulfate, filtered and solvent was distilled off. The residue was purified by means of silica gel column chromatography (hexane:ethyl acetate=2:1) to obtain 213. 6 mg of title compound being a mixture of four kinds of isomers (yield through two processes 50%).

Rf=0.48 (hexane:ethyl acetate=1:1).

HRMS(CI)(m/z): Calcd. for C₂₄H₄₀NO₆S [(M+diethanolamine)H⁺]: 470.2576. Found, 470.2560.

### EXAMPLE 12

(2R,6S)-Tert-butyl 2-[2-(E)-[dodecahydro-1-methoxynaphtho[2,3-c]furan-4-yl]ethenyl]-6-methylpiperidine-1-carboxylate

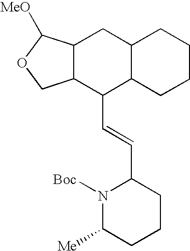

To 2 mL of a solution of 213.6 mg (0.59 mmol) of the compound of Example 11 in dehydrated dimethoxyethane were added dropwise 781.3 μL (1.17 mmol) of n-butyllithium-n-hexane solution (1.5 mol/L). After stirring for 5 minutes, 1 mL of a solution of 266.4 mg (1.17 mmol) of (2R,6S)-tert-butyl 2-formyl-6-methyl-1-piperidinecarboxylate in dehydrated dimethoxyethane was added dropwise. The mixture was stirred for 2 hours at the same temperature and 5 mL of water were added to the reaction mixture to stop the reaction, which was extracted with ether (3 ml×3). The organic layer was washed with brine (3 ml×3), dried over anhydrous magnesium sulfate, filtered and solvent was distilled off. The residue was purified by means of silica gel column chromatography (hexane:ethyl acetate=4:1, then 1:1) to obtain 272.3 mg of coupling resultant as a mixture of diastereomers.

To 10 mL of a solution of 272.3 mg of said coupling resultant in methanol were added 5.00 g of sodium amalgam (5%) and 1.00 g of disodium hydrogenphosphate at room temperature under stirring, and the mixture was stirred for 2 hours at room temperature. After 5 mL of water were added, the reaction mixture was filtered through celite, the residue was washed with ether (10 ml), and the organic layer was distilled off under reduced pressure. The residue was extracted with ether (3 ml×3). The organic layer was washed with brine (3 ml), dried over anhydrous magnesium sulfate, filtered and solvent was distilled off. The residue was purified by means of silica gel column chromatography (hexane:ethyl acetate=4 :1) to obtain 71.6 mg of title compound being a mixture of four kinds of isomers (yield through two processes 28%).

Rf=0.25 (hexane:ethyl acetate=4:1).

HRMS(CI)(m/z): Calcd. for C₂₆H₄₄NO₄ (MH⁺): 434.3270. Found, 434.3286.

### EXAMPLE 13

Dodecahydro-4-[2-(E)-[(2R,6S)-1,6-dimethylpiperidine-2-yl]ethenyl]naphtho[2,3-c]furan-1(3H)-one

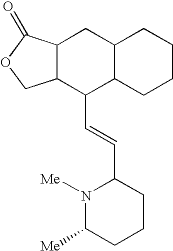

To 5 mL of a solution of 71.0 mg (0.16 mmol) of the compound of Example 12 in acetone was added 0.10 mL of Jones reagent at room temperature under stirring. After stirring for 2 hours at room temperature, solvent was distilled off and 5 mL of water was added to the residue, which was extracted with ether (3 ml×3). The organic layer was washed with brine (3 ml), dried over anhydrous magnesium sulfate, filtered and solvent was distilled off. The residue was further purified by means of silica gel column chromatography (hexane:ethyl acetate=1:1) to obtain 11.7 mg of (2R,6S)-tert-butyl 2-[2-(E)-[dodecahydro-1-oxonaphtho[2,3-c]furan-4-yl]ethenyl]-6-methylpiperidine-1-carboxylate (yield 17%).

To this compound was added 1 mL of methylene chloride for dissolution, and, after 0.50 mL of trifluoroacetic acid were added, the mixture was stirred for 1 hour at room temperature. After solvent was distilled off, 5 mL of cold dilute aqueous solution of sodium hydroxide were added to the residue to make alkaline, which was extracted with (chloroform:ethanol=10:1) (3 ml×3). The organic layer was dried over anhydrous magnesium sulfate, filtered and solvent was distilled off to obtain crude decahydro-4-[2-(E)-[(2R,6S)-6-methylpiperidine-2-yl]ethenyl]naphtho[2,3-c]furan-1(3H)-one.

To 2 mL of a solution of this compound in acetonitrile were added 0.50 mL of 37% aqueous formaldehyde and then 3.87 mg (61.6 μmol) of sodium cyanoborohydride at room temperature under stirring, and the mixture was stirred for 1.5 hours at room temperature. Acetic acid was added to the reaction mixture to make neutral and, after stirring further for 1.5 hours, 5 mL of cold dilute aqueous solution of sodium hydroxide were added to make alkaline and solvent was distilled off under reduced pressure. The residue was extracted with ether (3 ml×3). The organic layer was dried over anhydrous magnesium sulfate, filtered and solvent was distilled off. The residue was purified by means of silica gel column chromatography (silica gel NH, hexane:ethyl acetate=2:1) to obtain 3.20 mg of title compound being a mixture of four kinds of isomers (yield through two processes 34%).

Rf=0.07 (ethyl acetate:methanol=2:1).

HRMS(EI)(m/z): Calcd. for C₂₁H₃₃NO₂ (M⁺): 331.2511. Found, 331.2508.

Utilizability in the Industry

(Binding Test of Receptor)

For binding to muscarine M₁, cerebral cortex was used as a specimen and the evaluation was made with specific binding level of ³H-pyrenezepine. For the binding to muscarine M₂, brain stem and heart were used as specimens and the evaluation was made with specific binding level of ³H-quinuclidinyl benzylate. The nonspecific binding level was determined by using 1 μmol/L atropine. The concentration of radioligand was made to be 1 nmol/L for ³H-pyrenezepine and 0.5 nmol/L for ³H-quinuclidinyl benzylate.

Taking the membrane specimen and radioligand in a test tube, the total volume was made to be 1 mL with 50 mmol/L phosphate buffer. When determining the nonspecific binding level, 1 mol/L atropine was added and, when conducting the substitution experiment, testing compound thereof was added, respectively. Each drug etc. were diluted using 50 mmol/L phosphate buffer. After stirring well, the assay sample was incubated in a thermostatic shaker to perform the B/F separation using cell harvester. For the filter, GF/B was used. For performing this B/F separation, the filter was washed thrice with 5 mL of ice-cooled phosphate buffer. The radioactivity collected on the filter was meadured with scintillation counter using 10 mL of ACS-II.

As a result, the inventive compounds exhibited significant bondabilities to M₂ receptor. The inventive compounds are expected to be used as the therapeutic drugs for Alzheimer's disease.

1. Decahydronaphtho[2,3-c]furan derivatives represented by a following general formula (1) 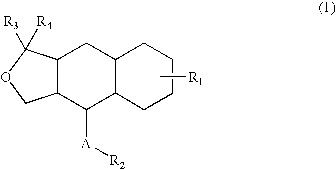
(wherein R₁ denotes a hydrogen atom, lower alkyl group or substituted or unsubstituted aralkyl group, R₂ denotes an aliphatic heterocycle of 5 to 14-membered ring which may contain one or more hetero-atoms such as O, N and S and which may have one or more substituents, or aromatic heterocycle, R₃ and R₄ are combined together to denote an oxygen atom, or R₃ denotes a hydrogen atom and R₄ denotes a hydroxyl group, lower alkoxy group, substituted or unsubstituted aralkyloxy group or lower acyloxy group, and A and R₂ unitedly denote following general formula (2) 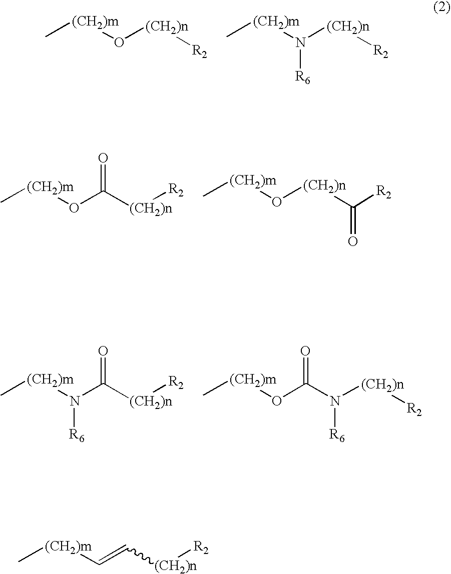
(m and n denote 0, 1 or 2, and R₅ denotes a hydrogen atom, lower alkyl group or substituted or unsubstituted aralkyl group)), and their acid addition salts.

2. Decahydronaphtho[2,3-c]furan derivatives represented by a following general formula (1-1) 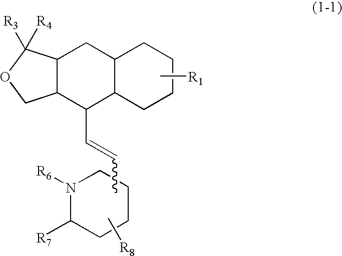
(wherein R₁ denotes a hydrogen atom, lower alkyl group or substituted or unsubstituted aralkyl group, R₃ and R₄ are combined together to denote an oxygen atom, or R₃ denotes a hydrogen atom and R₄ denotes a hydroxyl group, lower alkoxy group, substituted or unsubstituted aralkyloxy group or lower acyloxy group, R₆ denotes a hydrogen atom, lower alkyl group, substituted or unsubstituted aralkyl group or protective group of amino group, R₇ denotes a hydrogen atom, lower alkyl group or substituted or unsubstituted aralkyl group, and R₈ denotes a hydrogen atom, lower alkyl group or substituted or unsubstituted aralkyl group), and their acid addition salts.

3. Decahydronaphtho[2,3-c]furan derivatives represented by a following general formula (1-2) 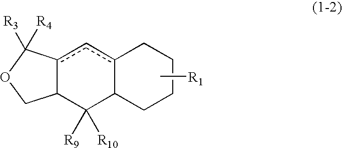
(wherein R₁ denotes a hydrogen atom, lower alkyl group or substituted or unsubstituted aralkyl group, R₃ and R₄ are combined together to denote an oxygen atom, or R₃ denotes a hydrogen atom and R₄ denotes a hydroxyl group, lower alkoxy group, substituted or unsubstituted aralkyloxy group or lower acyloxy group, R₉ and R₁₀ are combined together to denote an oxygen atom or methylene group, or R₉ denotes a hydrogen atom and R₁₀ denotes a hydroxyl group, lower alkoxy group, substituted or unsubstituted aralkyloxy group, lower acyloxy group, hydroxymethyl group which may be substituted with oxygen atom, substituted or unsubstituted arylsulfenylmethyl group or substituted or unsubstituted arylsulfonylmethyl group, and, in the case of broken lines accompanied, one denotes single bond and the other denotes double bond, or both denote single bonds).

4. Hydronaphtho[2,3-c]furan derivatives represented by a following general formula (3) 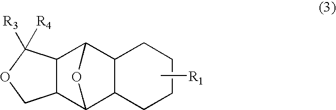
(wherein R₁ denotes a hydrogen atom, lower alkyl group or substituted or unsubstituted aralkyl group, R₃ and R₄ are combined together to denote an oxygen atom, or R₃ denotes a hydrogen atom and R₄ denotes a hydroxyl group, lower alkoxy group, substituted or unsubstituted aralkyloxy group or lower acyloxy group).

5. The compounds of claim 1, wherein one of them is decahydro-4-[2-(E)-[6-methylpiperidine-2-yl]ethenyl]naphtho[2,3-c]furan-1(3H)-one.

6. The compounds of claim 1, wherein one of them is decahydro-4-[2-(E)-[1,6-dimethylpiperidine-2-yl]ethenyl]naphtho [2,3-c]-furan-1(3H)-one.

7. An antagonist against muscarine M₂ subtype receptor, characterized by containing one or more kinds of decahydronaphtho[2,3-c]furan derivatives represented by a following general formula (1) 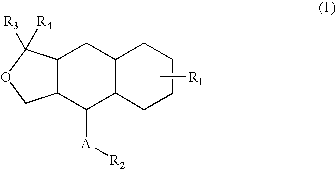
(wherein R₁ denotes a hydrogen atom, lower alkyl group or substituted or unsubstituted aralkyl group, R₂ denotes an aliphatic heterocycle of 5 to 14-membered ring which may contain one or more hetero-atoms such as O, N and S and which may have one or more substituents, or aromatic heterocycle, R₃ and R₄ are combined together to denote an oxygen atom, or R₃ denotes a hydrogen atom and R₄ denotes a hydroxyl group, lower alkoxy group, substituted or unsubstituted aralkyloxy group or lower acyloxy group, and A and R₂ are combined together to denote following general formula (2) 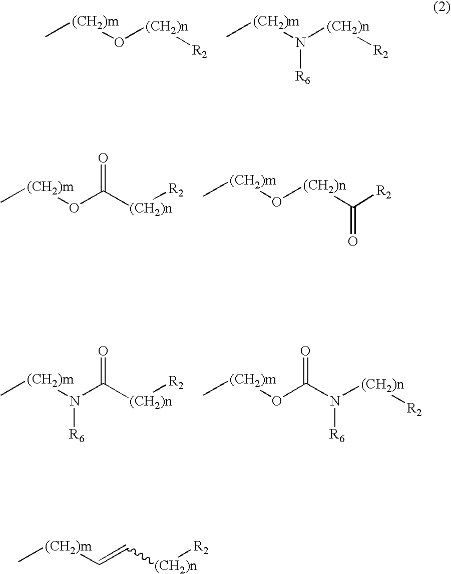
(m and n denote 0, 1 or 2, and R₅ denotes a hydrogen atom, lower alkyl group or substituted or unsubstituted aralkyl group)), and their acid addition salts, as effective ingredients.

8. An antagonist against muscarine M₂ subtype receptor, characterized by containing one or more kinds of decahydronaphtho[2,3-c]furan derivatives represented by a following general formula (1-1) 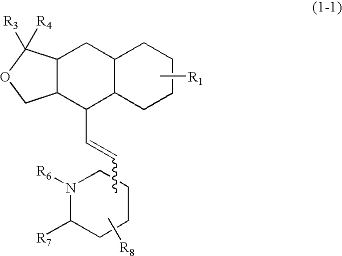
(wherein R₁ denotes a hydrogen atom, lower alkyl group or substituted or unsubstituted aralkyl group, R₃ and R₄ are combined together to denote an oxygen atom, or R₃ denotes a hydrogen atom and R₄ denotes a hydroxyl group, lower alkoxy group, substituted or unsubstituted aralkyloxy group or lower acyloxy group, R₆ denotes a hydrogen atom, lower alkyl group, substituted or unsubstituted aralkyl group or protective group of amino group, R₇ denotes a hydrogen atom, lower alkyl group or substituted or unsubstituted aralkyl group, and R₈ denotes a hydrogen atom, lower alkyl group or substituted or unsubstituted aralkyl group), and their acid addition salts, as effective ingredients.
